(19) United States
(12) Patent Application Publication
(10) Pub. No.: US 2025/0334545 A1
(43) Pub. Date: Oct. 30, 2025

(54) NANOFLUIDIC APPARATUS AND METHOD FOR MANIPULATING BIOMOLECULE

(71) Applicant: Quantum NIL Corporation, Taipei City (TW)

(72) Inventors: Wang-Nang WANG, Taichung CIty (TW); Chung-Hsiang LIN, Taipei City (TW)

(21) Appl. No.: 18/911,558

(22) Filed: Oct. 10, 2024

Related U.S. Application Data
(60) Provisional application No. 63/638,550, filed on Apr. 25, 2024.

(51) Int. Cl. G01N 27/447; C12Q 1/6869

(52) U.S. Cl. CPC G01N 27/44791; C12Q 1/6869

(57) ABSTRACT

A nanofluidic apparatus for manipulating a biomolecule includes a substrate, an actuator, and a nanochannel. The actuator is connected to the substrate so as to cause repetitive expansion and contraction of the substrate. The nanochannel is formed in the substrate, and includes an inner surface and nano features formed on the inner surface. The nanochannel has an inlet end for introducing the biomolecule into the nanochannel, and an outlet end opposite to the inlet end. In response to the repetitive expansion and contraction of the substrate, the biomolecule is stretched by the nano features into a linearized form and is driven by the nano features to move toward the outlet end. A method for manipulating a biomolecule is also disclosed.

### CROSS-REFERENCE TO RELATED APPLICATION

This application claims the benefit of U.S. Provisional Patent Application No. 63/638,550, filed on Apr. 25, 2024, the entire disclosure of which is incorporated by reference herein.

### FIELD

The present disclosure relates to a nanofluidic apparatus for manipulating a biomolecule. The present disclosure also relates to a method for manipulating a biomolecule.

### BACKGROUND

In the field of biomolecular analysis, various nanopore-based devices have been developed for biopolymer sequencing; however, the dynamics of biopolymer movement through the nanopore of these devices is difficult to be controlled, for example, the speed of biopolymer, e.g. DNA, RNA, etc., moving through the nanopore is too fast, making it impossible to discriminate the nucleotides along a DNA strand. Hence, most of the conventional nanopore-based devices lack the ability to accurately and precisely analyze biopolymer.

### SUMMARY

Therefore, an object of the present disclosure is to provide a nanofluidic apparatus and a method for manipulating a biomolecule that can alleviate at least one of the drawbacks of the prior art.

According to an aspect of the present disclosure, the nanofluidic apparatus for manipulating a biomolecule includes a substrate, an actuator, and a nanochannel. The actuator is connected to the substrate so as to cause repetitive expansion and contraction of the substrate along a Y direction. The nanochannel is formed in the substrate, and includes an inner surface and nano features that are formed on the inner surface. The nanochannel has an inlet end for introducing the biomolecule into the nanochannel, and an outlet end opposite to the inlet end in the Y direction. In response to the repetitive expansion and contraction of the substrate, the biomolecule is stretched by the nano features into a linearized form and is driven by the nano features to move toward the outlet end.

According to another aspect of the present disclosure, the method for manipulating a biomolecule includes the steps of:
a) introducing the biomolecule into a nanochannel inside a substrate through an inlet end of the nanochannel, the nanochannel including an inner surface and nano features formed on the inner surface; and
b) subjecting the substrate to repetitive expansion and contraction along a Y direction such that the biomolecule is stretched by the nano features into a linearized form and is driven by the nano features to move toward an outlet end of the nanochannel which is opposite to the inlet end in the Y direction.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 is a schematic perspective view illustrating a nanofluidic apparatus for manipulating a biomolecule according to certain embodiments of the present disclosure.

FIG. 2 is a schematic sectional view taken along line A-A′ of FIG. 1 according to some embodiments of the present disclosure.

FIG. 3A is a partially enlarged schematic view of an area (B) of FIG. 2 illustrating nano features having a same length, and a same slanted degree according to some embodiments of the present disclosure.

FIG. 3B is a view similar to FIG. 3A but illustrating the nano features having different slanted degrees according to some embodiments of the present disclosure.

FIGS. 4A and 4B are views respectively similar to FIGS. 3A and 3B but illustrating the nano features having different lengths according to some embodiments of the present disclosure.

FIG. 5 is a schematic sectional view taken along line C-C′ of FIG. 3A according to some embodiments of the present disclosure.

### DETAILED DESCRIPTION

Before the present disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

It should be noted herein that the drawings, which are shown for illustrative purposes only, are not drawn to scale, and are not intended to represent the actual sizes or actual relative sizes of the features/components illustrated in the drawings.

In order to address the current limitations of nanopore-based devices for sequencing of biomolecules, the inventors of this application endeavored to developing a nanofluidic apparatus including a substrate, an actuator, and an active nanochannel template including slanted nano features, such that upon repetitive contraction and expansion of the substrate driven by the actuator, the slanted nanofeatures are capable of stretching the biomolecule into a linearized form in the active nanochannel template so as to permit the linearized biomolecule to pass through the active nanochannel template under a well-controlled speed, thereby prolonging the retention time of the linearized biomolecule in the active nanochannel template for accurate identification thereof.

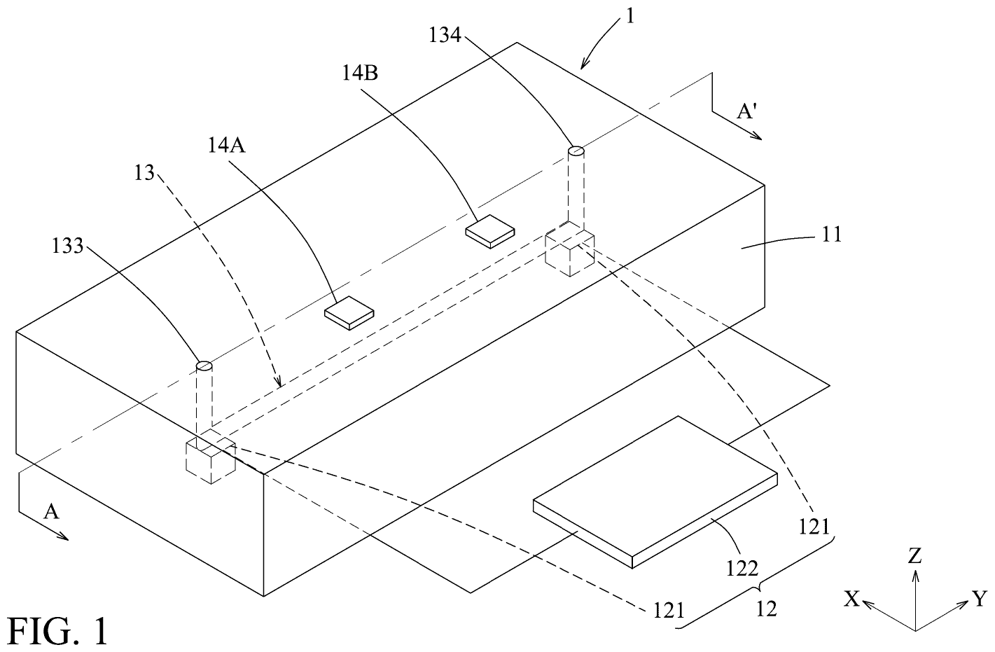

Referring to FIG. 1, a nanofluidic apparatus 1 for manipulating a biomolecule (not shown) according to an embodiment of the present disclosure includes a substrate 11, an actuator 12 connected to the substrate 11, and a nanochannel 13 formed in the substrate 11.

In some embodiments, the substrate 11 may be made of a piezoelectric material. Examples of the piezoelectric material may include, but are not limited to, lead zirconate titanate (PbZrTiO₃), zinc oxide (ZnO), gallium nitride (GaN), polyvinylidene fluoride (PVDF), barium titanate (BaTiO₃), sodium potassium niobate (KNaNbO₃), quartz, ceramic composites, berlinite (AlPO₄), lead titanate (PbTiO₃), lithium niobate (LiNbO₃), lithium tantalite (LiTaO₃), sodium tungstate (Na₂WO₃), bismuth ferrite (BiFeO₃), bismuth titanate (Bi₄Ti₃O₁₂), and boron nitride (BN). In other embodiments, the substrate 11 may be light-transmissive.

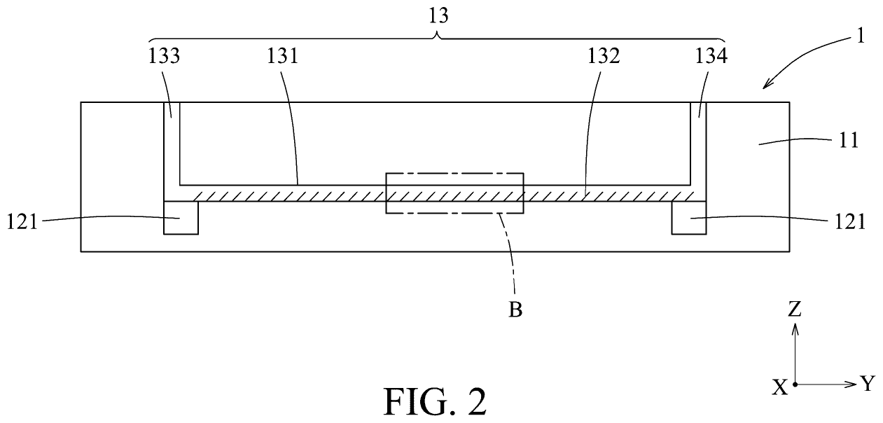

According to the present disclosure, as shown in FIGS. 1 and 2, the nanochannel 13 includes an inner surface 131 and nano features 132 that are formed on the inner surface 131, and has an inlet end 133 for introducing the biomolecule into the nanochannel 13 and an outlet end 134 opposite to the inlet end 133 in a Y direction. In some embodiments, a solution, for example, a low ionic strength buffer solution, which includes the biomolecule and a suitable additive, such as, dye-labeled nucleotides, dideoxynucleotides, fluorophore-labeled reversible terminator nucleotides, etc., is introduced into the nanochannel 13.

In certain embodiments, the actuator 12 includes two electrodes 121 and a pulse generator 122 connected to the two electrodes 121. The two electrodes 121 are formed in the substrate 11 and located opposite to each other in the Y direction. In some embodiments, the two electrodes 121 may be formed beneath the nanochannel 13 and located opposite to each other in the Y direction. The pulse generator 122 is capable of generating an adjustable pulse-width modulation (PWM) signal, causing repetitive expansion and contraction of the substrate 11 along the Y direction, such that the biomolecule introduced into the nanochannel 13 is stretched by the nano features 132 into a linearized form and is driven by the nano features 132 to move toward the outlet end 134.

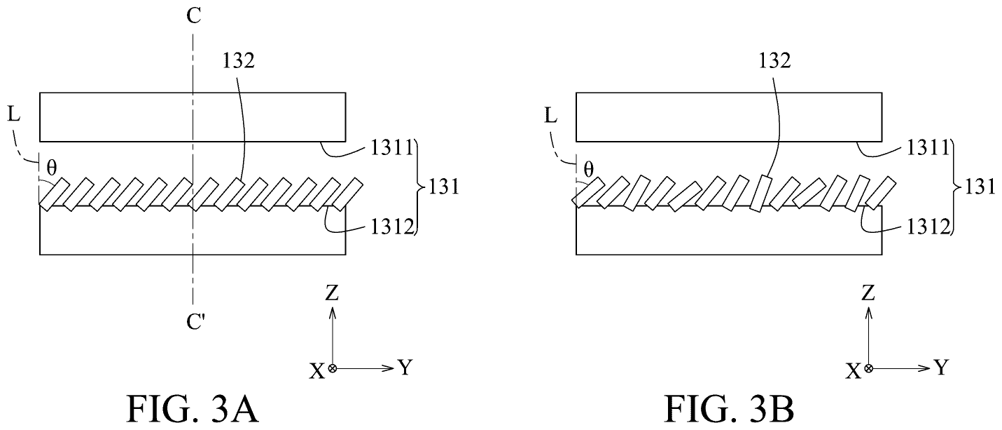

Referring to FIG. 3A, in certain embodiments, each of the nano features 132 extends from the inner surface 131 of the nanochannel 13 and slants relative to a reference line (L) toward the outlet end 134 by a slanted degree (θ), i.e., an angle formed between the reference line (L) and the slanted side of each of the nano features 132. In this case, the reference line (L) is arranged normal to the inner surface 131, and the slanted degree (θ) ranges from greater than 0° and not greater than 60°.

According to the present disclosure, the nanofeatures 132 may be formed with different configurations. As shown in FIG. 3A, the nano features 132 have a same length, and the slanted degrees (θ) of the nano features 132 are the same. In certain embodiments, as shown in FIG. 3B, the nano features 132 have a same length, and the slanted degrees (θ) of the nano features 132 on different regions of the nanochannel 13 are different.

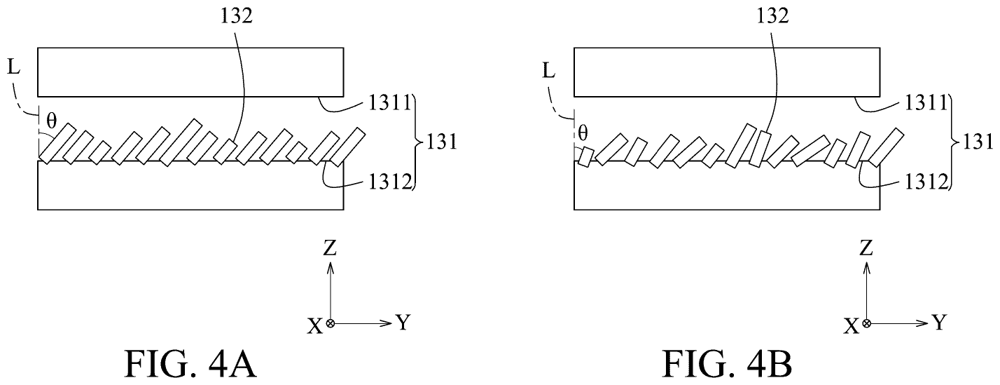

Referring to FIG. 4A, in certain embodiments, the nano features 132 on different regions of the nanochannel 13 have different lengths, and the slanted degrees (θ) of the nano features 132 are the same. In other embodiments, as shown in FIG. 4B, the nano features 132 on different regions of the nanochannel 13 have different lengths, and the slanted degrees (θ) of the nano features 132 on different regions of the nanochannel 13 are different.

In certain embodiments, the nano features 132 are slanted nanorods.

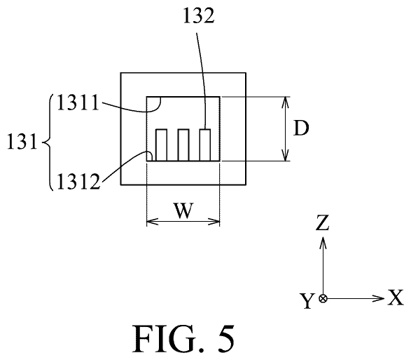

According to the present disclosure, as shown in FIG. 5, the inner surface 131 of the nanochannel 13 includes an upper part 1311 and a lower part 1312 opposite to the upper part 1311 in a Z direction which is transverse to the Y direction, and the nano features 132 are formed on at least one of the upper part 1311 and the lower part 1312 of the inner surface 131. That is, in certain embodiments, the nano features 132 are formed on the lower part 1312 (see FIG. 5), while in other embodiments, the nano features 132 are formed on the upper part 1311 or on both the upper part 1311 and the lower part 1312 (not shown in the figures).

In certain embodiments, the upper part 1311 and the lower part 1312 are spaced apart from each other by a distance (D) that ranges from 2 nm to 100 nm in the Z direction (see FIG. 5).

In certain embodiments, each of the upper part 1311 and the lower part 1312 has a width (W) ranging from 2 nm to 100 nm in an X direction transverse to both the Y direction and the Z direction (see FIG. 5).

In certain embodiments, the nano features 132 are arranged in columns and rows in the Y direction and the X direction transverse to both the Y direction and the Z direction.

In some embodiments, the nanofluidic apparatus 1 may be formed by: preparing an upper substrate portion and a lower substrate portion; forming two through holes in the upper substrate portions, the two through holes respectively serving as the inlet end 133 and the outlet end 134; forming the upper part 1311 of the inner surface 131 in the upper substrate portion using a suitable fabrication process or other suitable processes; forming the lower part 1312 of the inner surface 131 in the lower substrate portion using a suitable fabrication process or other suitable processes; bonding the upper substrate portion with the lower substrate portion to form the substrate 11 so that the nanochannel 13 with the nanofeatures 132 is formed in the substrate 11; forming the two electrodes 121 beneath the substrate 11; and electrically connecting the two electrodes 121 to the pulse generator 122. The nano features 132 may be formed separately or together with the inner surface 131 of the nanochannel 13. In the case that the nano features 132 are formed on the lower part 1312 of the inner surface 131, during formation of the lower part 1312, directional plasma treatment processes, reactive ion beam etching (RIBE), chemically assisted ion beam etching (CAIBE) or other suitable processes may be utilized for forming the nano features 132 on the lower part 1312. Alternatively, after forming the lower part 1312, the nano features 132 are formed on the lower part 1312 using three-dimensional printing processes or other suitable processes.

According to the present disclosure, nano features 132 may be coated with positively-charged molecules or other molecules (e.g., hydrophobic or hydrophilic molecules) capable of selectively interacting with the biomolecule. Examples of the positively-charged molecules or other molecules may include radioactive or fluorescent dyes, but are not limited thereto. The positively-charged molecules or other molecules are capable of forming van der Waals bonds, dipole moments, coulombic force, or charge transfer across the nano features 132, so as to facilitate stretching and movement of the biomolecule in the nanochannel 13 along the Y direction.

Referring again to FIG. 1, in certain embodiments, the nanofluidic apparatus 1 further includes a detector 14A and a detector 14B that are disposed on the substrate 11, and that are capable of recognizing segments of the biomolecule in the linearized form when the segments of the biomolecule sequentially pass through the nanochannel 13 beneath the detector 14A and the detector 14B. By inclusion of both the detector 14A and the detector 14B, recognition of segments of the biomolecule would be made twice, i.e., a first recognition is conducted by the detector 14A and then a second recognition conducted by the detector 14B, so as to enhance the detection accuracy. In other embodiments, the nanofluidic apparatus 1 includes only one of the detector 14A and the detector 14B.

In other embodiments, the detector 14A and/or detector 14B are placed adjacent and connected to the substrate 11 that is light-transmissive to recognize segments of the biomolecule in the linearized form.

Examples of the detector 14A and the detector 14B may include, but are not limited to, laser scanner, total internal reflection fluorescence microscope, Raman spectrophotometer, optical tweezers, and chemiluminescence analyzer.

It is noted that, since the substrate 11 is made of a piezoelectric material and since the inner surface 131 of the nanochannel 13 of the nanofluidic apparatus 1 is formed with nano features 132, upon repetitive expansion and contraction of the substrate 11, a biomolecule in the nanochannel 13 can be stretched into a linearized form and be driven by the nano features 132 to move toward the outlet end 134 of the nanochannel 13 under a well-controlled speed (i.e., the retention time of the biomolecule in the nanochannel 13 is sufficient to permit detailed analysis of the biomolecule), and thus the nanofluidic apparatus 1 is deemed useful for analyzing the biomolecule.

Therefore, the present disclosure also provides a method for manipulating a biomolecule using the nanofluidic apparatus 1. The method includes step a) and b). In step a), the biomolecule dispersed in the solution, e.g., the low ionic strength buffer solution, is introduced into the nanochannel 13 inside the substrate 11 through the inlet end 133. In step b), the substrate 11 is subjected to repetitive expansion and contraction along the Y direction such that the biomolecule is stretched by the nano features 132 into a linearized form and is driven by the nano features 132 to move toward the outlet end 134 of the nanochannel 13 which is opposite to the inlet end 133 in the Y direction.

According to the present disclosure, in step b), the pulse-width modulation (PWM) signal from the pulse generator 122 is applied to the substrate 11 so as to drive the repetitive expansion and movement of the substrate 11 along the Y direction.

In certain embodiments, in step b), a displacement speed of the biomolecule in the nanochannel 13 is varied by adjusting the PWM signal. For instance, when the PWM signal has a lower switching frequency, a lower power, and/or a lower amplitude, the biomolecule in the nanochannel 13 has a relatively lower displacement speed. In addition, when the biomolecule is fully stretched into the linearized form, the PWM signal may be halted so that the biomolecule may remain in the nanochannel 13 to allow identification of the biomolecule for a sufficient period of time.

According to the present disclosure, the biomolecule is a polynucleotide. Examples of the polynucleotide include, DNA molecule (e.g., single-strand DNA molecule and double-strand DNA molecule), and RNA molecule, but are not limited thereto. In certain embodiments, the polynucleotide is a single-strand DNA molecule. In other embodiments, the polynucleotide is a double-strand DNA molecule.

According to the present disclosure, the method further includes detecting nucleobases of the DNA molecule that is present in the linearized form, which sequentially pass through a first predetermined position in the nanochannel 13. It should be noted that, in certain embodiments, the first predetermined position of the DNA molecule in the nanochannel 13 corresponds to the position where the detector 14A is disposed on the substrate 11.

In certain embodiments, the method further includes detecting nucleobases of the DNA molecule that is present in the linearized form, which sequentially pass through a second predetermined position in the nanochannel 13 that is located downstream of the first predetermined position, and then determining a sequence of the DNA molecule by comparing the nucleobases of the DNA molecule detected at the first predetermined position and the second predetermined position. In this case, the first predetermined position and the second predetermined position of the DNA molecule in the nanochannel 13 respectively corresponds to the positions where the detector 14A and the detector 14B are respectively disposed on the substrate 11. It should be noted that, since the distance between the first predetermined position and the second predetermined position can be determined based on a distance between the detector 14A and the detector 14B, and since the PWM signal can be adjusted to adjust the displacement speed of the DNA molecule such that a time period taken for each of the nucleobases of the DNA molecule to move from the first predetermined position to the second predetermined position is constant, detection of the nucleobases of the DNA molecule in the nanochannel 13 is conducted twice by the detector 14A and the detector 14B at the first predetermined position and the second predetermined position, respectively, thereby enhancing the accuracy of the thus determined sequence of the DNA molecule, i.e., minimizing error rate of the same.

In summary, by virtue of the method for manipulating a biomolecule of the present disclosure which utilizes the nanofluidic apparatus 1, in response to the repetitive expansion and contraction of the substrate 11, the biomolecule, e.g., a DNA molecule, can be stretched by the nano features 132 into a linearized form and be driven by the nano features 132 to move through the nanochannel 13 at a relatively slow speed, i.e., the retention time of the DNA molecule in the nanochannel 13 is sufficient to allow identification of each of the nucleobases of the DNA molecule by the detector 14A and/or detector 14B, so as to determine the sequence of the DNA molecule, and hence detailed and accurate analysis of the DNA molecule can be achieved.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A nanofluidic apparatus for manipulating a biomolecule, comprising:
a substrate;
an actuator connected to said substrate so as to cause repetitive expansion and contraction of said substrate along a Y direction; and
a nanochannel formed in said substrate, and including an inner surface and nano features that are formed on said inner surface, said nanochannel having
an inlet end for introducing the biomolecule into said nanochannel, and
an outlet end opposite to said inlet end in the Y direction,
wherein in response to the repetitive expansion and contraction of said substrate, the biomolecule is stretched by said nano features into a linearized form and is driven by said nano features to move toward said outlet end.

2. The nanofluidic apparatus as claimed in claim 1, wherein said substrate is made of a piezoelectric material, and said actuator includes
two electrodes formed in said substrate and located opposite to each other in the Y direction, and
a pulse generator connected to said two electrodes, and being capable of generating an adjustable pulse-width modulation (PWM) signal so as to cause repetitive expansion and contraction of said substrate.

3. The nanofluidic apparatus as claimed in claim 2, wherein said piezoelectric material is selected from the group consisting of lead zirconate titanate (PbZrTiO₃), zinc oxide (ZnO), gallium nitride (GaN), polyvinylidene fluoride (PVDF), barium titanate (BaTiO₃), sodium potassium niobate (KNaNbO₃), quartz, ceramic composites, berlinite (AlPO₄), lead titanate (PbTiO₃), lithium niobate (LiNbO₃), lithium tantalite (LiTaO₃), sodium tungstate (Na₂WO₃), bismuth ferrite (BiFeO₃), bismuth titanate (Bi₄Ti₃O₁₂), boron nitride (BN), and combinations thereof.

4. The nanofluidic apparatus as claimed in claim 1, wherein each of said nano features extends from said inner surface of said nanochannel, and slants relative to a reference line toward said outlet end by a slanted degree,
the reference line being arranged normal to said inner surface, and
the slanted degree ranging from greater than 0° and not greater than 60°.

5. The nanofluidic apparatus as claimed in claim 4, wherein said slanted degrees of said nano features are the same.

6. The nanofluidic apparatus as claimed in claim 4, wherein the slanted degrees of said nano features on different regions of said nanochannel are different.

7. The nanofluidic apparatus as claimed in claim 1, wherein said nano features have a same length.

8. The nanofluidic apparatus as claimed in claim 1, wherein said nano features on different regions of said nanochannel have different lengths.

9. The nanofluidic apparatus as claimed in claim 1, wherein
said inner surface of said nanochannel includes an upper part and a lower part opposite to said upper part in a Z direction which is transverse to the Y direction, and
said nano features are formed on at least one of said upper part and said lower part of said inner surface.

10. The nanofluidic apparatus as claimed in claim 9, wherein said upper part and said lower part are spaced apart from each other by a distance ranging from 2 nm to 100 nm.

11. The nanofluidic apparatus as claimed in claim 9, wherein each of said upper part and said lower part has a width in an X direction transverse to both the Y direction and the Z direction, said width ranging from 2 nm to 100 nm.

12. The nanofluidic apparatus as claimed in claim 9, wherein said nano features are arranged in columns and rows in the Y direction and an X direction transverse to both the Y direction and the Z direction.

13. The nanofluidic apparatus as claimed in claim 1, wherein said nano features are coated with positively-charged molecules or other molecules capable of selectively interacting with the biomolecule.

14. The nanofluidic apparatus as claimed in claim 1, wherein said substrate is light-transmissive, the nanofluidic apparatus further comprising a detector that is disposed on said substrate and that is capable of recognizing segments of the biomolecule in the linearized form when the segments of the biomolecule sequentially pass through said nanochannel beneath said detector.

15. The nanofluidic apparatus as claimed in claim 1, wherein said nano features are slanted nanorods.

16. A method for manipulating a biomolecule, comprising steps of:
a) introducing the biomolecule into a nanochannel inside a substrate through an inlet end of the nanochannel, the nanochannel including an inner surface and nano features formed on the inner surface; and
b) subjecting the substrate to repetitive expansion and contraction along a Y direction such that the biomolecule is stretched by the nano features into a linearized form and is driven by the nano features to move toward an outlet end of the nanochannel which is opposite to the inlet end in the Y direction.

17. The method as claimed in claim 16, wherein
the substrate is made of a piezoelectric material, and
in step b), a pulse-width modulation (PWM) signal is applied to the substrate so as to drive the repetitive expansion and contraction of the substrate along the Y direction.

18. The method as claimed in claim 17, wherein in step b), a displacement speed of the biomolecule in the nanochannel is varied by adjusting the PWM signal.

19. The method as claimed in claim 16, wherein the biomolecule is a DNA molecule, the method further comprising:
detecting nucleobases of the DNA molecule in the linearized form, which sequentially pass through a first predetermined position in the nanochannel.

20. The method as claimed in claim 19, further comprising:
detecting the nucleobases of the DNA molecule in the linearized form, which sequentially pass through a second predetermined position in the nanochannel that is located downstream of the first predetermined position; and
determining a sequence of the DNA molecule by comparing the nucleobases of the DNA molecule detected at the first predetermined position and the second predetermined position.
